(19) United States
(12) Patent Application Publication
(10) Pub. No.: US 2005/0186577 A1
(43) Pub. Date: Aug. 25, 2005

(54) Breast cancer prognostics

(76) Inventor: Yixin Wang, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/783,271

(22) Filed: Feb. 20, 2004

(51) Int. Cl. C12Q 1/68

(52) U.S. Cl. 435/6

(57) ABSTRACT

A method of providing a prognosis of breast cancer is conducted by analyzing the expression of a group of genes. Gene expresson profiles in a variety of medium such as microarrays are included as are kits that contain them.

### BACKGROUND

This invention relates to prognostics for breast cancer based on the gene expression profiles of biological samples.

Breast cancer is a heterogeneous disease that exhibits a wide variety of clinical presentations, histological types and growth rates. Because of these variations, determining prognosis for an individual patient at the time of initial diagnosis requires careful assessment of multiple clinical and pathological parameters, but the currently used traditional prognostic factors are not sufficient. In primary breast cancer, metastasis to axillary lymph nodes is the most important clinical prognostic factor. Approximately 60% of lymph-node-negative (LNN) patients are cured by local-regional treatment alone. Many patients that relapse eventually die due to resistance to systemic endocrine or chemotherapy given as treatment for recurrent disease. It is particularly important to identify the LNN patients that are at high risk for relapse since they generally need adjuvant systemic therapy after primary surgery. It would also be beneficial to more confidently be able to avoid administering adjuvant therapy to LNN patients that do not require it.

Currently in LNN patients, the decision to apply adjuvant therapy or not after surgical removal of the primary tumor, and which type (endocrine- and/or chemotherapy), largely depends on patient's age, menopausal status, tumor size, tumor grade, and the steroid hormone-receptor status. These factors are accounted for in guidelines such as St. Gallen criteria and the National Institutes of Health (NIH) consensus criteria. Based on these criteria more than 85%-90% of the LNN patients would be candidates to receive adjuvant systemic therapy.

There is clearly a need to identify better prognostic factors for guiding selection of treatment choices.

### SUMMARY OF THE INVENTION

The invention is a method of assessing the likelihood of a recurrence of breast cancer in a patient diagnosed with or treated for breast cancer. The method involves the analysis of a gene expression profile made up of a combination of genes from the genes found in SEQ ID NO 36-111.

In one aspect of the invention, the gene expression profile includes at least 35 genes (SEQ ID NO 1-35).

In another aspect of the invention, the gene expression profile includes at least 60 particular genes (SEQ ID NO 36-95). This profile is particularly useful in prognosticating ER positive patients.

In another aspect of the invention, the gene expression profile includes at least 16 particular genes (SEQ ID NO 96-111). This profile is particularly useful in prognosticating ER negative patients.

In another aspect of the invention, the gene expression profile includes at least 76 particular genes (SEQ ID NO 36-111).

Articles used in practicing the methods are also an aspect of the invention. Such articles include gene expression profiles or representations of them that are fixed in machine-readable media such as computer readable media.

Articles used to identify gene expression profiles can also include substrates or surfaces, such as microarrays, to capture and/or indicate the presence, absence, or degree of gene expression.

In yet another aspect of the invention, kits include reagents for conducting the gene expression analysis prognostic of breast caner recurrence.

### BRIEF DESCRIPTION OF THE DRAWINGS

FIG. 1 is a Receiver Operator Curve (ROC) produced using the 171 patients in the testing set and used AUC to assess the performance of the 76 gene signature.

FIG. 2 is a standard Kaplan-Meier Plot constructed for distant metastasis free survival (DMFS) as a function of the 76 gene-signature. The vertical axis shows the probability of disease-free survival among patients in each class.

FIG. 3 is a standard Kaplan-Meier Plot constructed for overall survival (OS) as a function of the 76 gene-signature. The vertical axis shows the probability of disease-free survival among patients in each class.

### DETAILED DESCRIPTION

The mere presence or absence of particular nucleic acid sequences in a tissue sample has only rarely been found to have diagnostic or prognostic value. Information about the expression of various proteins, peptides or mRNA, on the other hand, is increasingly viewed as important. The mere presence of nucleic acid sequences having the potential to express proteins, peptides, or mRNA ( such sequences referred to as “genes”) within the genome by itself is not determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression can provide useful information about the occurrence of important events such as tumerogenesis, metastasis, apoptosis, and other clinically relevant phenomena. Relative indications of the degree to which genes are active or inactive can be found in gene expression profiles. The gene expression profiles of this invention are used to provide a prognosis and treat patients for breast cancer.

Sample preparation requires the collection of patient samples. Patient samples used in the inventive method are those that are suspected of containing diseased cells such as epithelial cells taken from the primary tumor in a breast sample. Samples taken from surgical margins are also preferred. Most preferably, however, the sample is taken from a lymph node obtained from a breast cancer surgery. Laser Capture Microdisection (LCM) technology is one way to select the cells to be studied, minimizing variability caused by cell type heterogeneity. Consequently, moderate or small changes in gene expression between normal and cancerous cells can be readily detected. Samples can also comprise circulating epithelial cells extracted from peripheral blood. These can be obtained according to a number of methods but the most preferred method is the magnetic separation technique described in U.S. Pat. No. 6,136,182 (assigned to Immunivest Corporation) which is incorporated herein by reference. Once the sample containing the cells of interest has been obtained, RNA is extracted and amplified and a gene expression profile is obtained, preferably via micro-array, for genes in the appropriate portfolios.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complimentary DNA (cDNA) or complimentary RNA (cRNA) produced from mRNA and analyze it via microarray. A number of different array configurations and methods for their production are known to those of skill in the art and are described in U.S. Pat. Nos. such as: 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637; the disclosures of which are incorporated herein by reference.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. No. 6,271,002 to Linsley, et al.; U.S. Pat. No. 6,218,122 to Friend, et al.; U.S. Pat. No. 6,218,114 to Peck, et al.; and U.S. Pat. No. 6,004,755 to Wang, et al., the disclosure of each of which is incorporated herein by reference.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from normal tissue of the same type (e.g., diseased breast tissue sample vs. normal breast tissue sample). A ratio of these expression intensities indicates the fold-change in gene expression between the test and control samples.

Gene expression profiles can also be displayed in a number of ways. The most common method is to arrange raw fluorescence intensities or ratio matrix into a graphical dendogram where columns indicate test samples and rows indicate genes. The data is arranged so genes that have similar expression profiles are proximal to each other. The expression ratio for each gene is visualized as a color. For example, a ratio less than one (indicating down-regulation) may appear in the blue portion of the spectrum while a ratio greater than one (indicating up-regulation) may appear as a color in the red portion of the spectrum. Commercially available computer software programs are available to display such data including “GENESPRING” from Silicon Genetics, Inc. and “DISCOVERY” and “INFER” software from Partek, Inc.

Modulated genes used in the methods of the invention are described in the Examples. The genes that are differentially expressed are either up regulated or down regulated in patients with a relapse of colon cancer relative to those without a relapse. Up regulation and down regulation are relative terms meaning that a detectable difference (beyond the contribution of noise in the system used to measure it) is found in the amount of expression of the genes relative to some baseline. In this case, the baseline is the measured gene expression of a non-relapsing patient. The genes of interest in the diseased cells (from the relapsing patients) are then either up regulated or down regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis includes the determination of disease/status issues such as determining the likelihood of relapse and therapy monitoring. In therapy monitoring, clinical judgments are made regarding the effect of a given course of therapy by comparing the expression of genes over time to determine whether the gene expression profiles have changed or are changing to patterns more consistent with normal tissue.

Preferably, levels of up and down regulation are distinguished based on fold changes of the intensity measurements of hybridized microarray probes. A 2.0 fold difference is preferred for making such distinctions (or a p-value less than 0.05). That is, before a gene is said to be differentially expressed in diseased/relapsing versus normal/non-relapsing cells, the diseased cell is found to yield at least 2 times more, or 2 times less intensity than the normal cells. The greater the fold difference, the more preferred is use of the gene as a diagnostic or prognostic tool. Genes selected for the gene expression profiles of the instant invention have expression levels that result in the generation of a signal that is distinguishable from those of the normal or non-modulated genes by an amount that exceeds background using clinical laboratory instrumentation.

Statistical values can be used to confidently distinguish modulated from non-modulated genes and noise. Statistical tests find the genes most significantly different between diverse groups of samples. The Student's t-test is an example of a robust statistical test that can be used to find significant differences between two groups. The lower the p-value, the more compelling the evidence that the gene is showing a difference between the different groups. Nevertheless, since microarrays measure more than one gene at a time, tens of thousands of statistical tests may be asked at one time. Because of this, one is unlikely to see small p-values just by chance and adjustments for this using a Sidak correction as well as a randomization/permutation experiment can be made. A p-value less than 0.05 by the t-test is evidence that the gene is significantly different. More compelling evidence is a p-value less then 0.05 after the Sidak correction is factored in. For a large number of samples in each group, a p-value less than 0.05 after the randomization/permutation test is the most compelling evidence of a significant difference.

Another parameter that can be used to select genes that generate a signal that is greater than that of the non-modulated gene or noise is the use of a measurement of absolute signal difference. Preferably, the signal generated by the modulated gene expression is at least 20% different than those of the normal or non-modulated gene (on an absolute basis). It is even more preferred that such genes produce expression patterns that are at least 30% different than those of normal or non-modulated genes.

Genes can be grouped so that information obtained about the set of genes in the group provides a sound basis for making a clinically relevant judgment such as a diagnosis, prognosis, or treatment choice. These sets of genes make up the portfolios of the invention. In this case, the judgments supported by the portfolios involve breast cancer and its chance of recurrence. As with most diagnostic markers, it is often desirable to use the fewest number of markers sufficient to make a correct medical judgment. This prevents a delay in treatment pending further analysis as well inappropriate use of time and resources.

Preferably, portfolios are established such that the combination of genes in the portfolio exhibit improved sensitivity and specificity relative to individual genes or randomly selected combinations of genes. In the context of the instant invention, the sensitivity of the portfolio can be reflected in the fold differences exhibited by a gene's expression in the diseased state relative to the normal state. Specificity can be reflected in statistical measurements of the correlation of the signaling of gene expression with the condition of interest. For example, standard deviation can be a used as such a measurement. In considering a group of genes for inclusion in a portfolio, a small standard deviation in expression measurements correlates with greater specificity. Other measurements of variation such as correlation coefficients can also be used in this capacity.

One method of establishing gene expression portfolios is through the use of optimization algorithms such as the mean variance algorithm widely used in establishing stock portfolios. This method is described in detail in the patent application entitled “Portfolio Selection” by Tim Jatkoe, et. al., filed on Mar. 21, 2003. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application”, referred to as “Wagner Software” throughout this specification, is preferred. This software uses functions from the “Wagner Associates Mean-Variance Optimization Library” to determine an efficient frontier and optimal portfolios in the Markowitz sense is preferred. Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with breast cancer. Output from the method would be an optimized set of genes that could include some genes that are expressed in peripheral blood as well as in diseased tissue. If samples used in the testing method are obtained from peripheral blood and certain genes differentially expressed in instances of breast cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

One method of the invention involves comparing gene expression profiles for various genes (or portfolios) to ascribe prognoses. The gene expression profiles of each of the genes comprising the portfolio are fixed in a medium such as a computer readable medium. This can take a number of forms. For example, a table can be established into which the range of signals (e.g., intensity measurements) indicative of disease is input. Actual patient data can then be compared to the values in the table to determine whether the patient samples are normal or diseased. In a more sophisticated embodiment, patterns of the expression signals (e.g., flourescent intensity) are recorded digitally or graphically. The gene expression patterns from the gene portfolios used in conjunction with patient samples are then compared to the expression patterns. Pattern comparison software can then be used to determine whether the patient samples have a pattern indicative of recurrence of the disease. Of course, these comparisons can also be used to determine whether the patient is not likely to experience disease recurrence. The expression profiles of the samples are then compared to the portfolio of a control cell. If the sample expression patterns are consistent with the expression pattern for recurrence of a breast cancer then (in the absence of countervailing medical considerations) the patient is treated as one would treat a relapse patient. If the sample expression patterns are consistent with the expression pattern from the normal/control cell then the patient is diagnosed negative for breast cancer.

The preferred profiles of this invention are the 35-gene portfolio made up of the genes of SEQ ID NO 1-35, the 60-gene portfolio made up of the genes of SEQ ID NO 36-95 which is best used to prognosticate ER positive patients, and the 16-gene portfolio made up of genes of SEQ ID NO 96-111 which is best used to prognosticate ER negative patients. Most preferably, the portfolio is made up of genes of SEQ ID NO 36-111. This most preferred portfolio best segregates breast cancer patients irrespective of ER status at high risk of relapse from those who are not. Once the high-risk patients are identified they can then be treated with adjuvant therapy.

In this invention, the most preferred method for analyzing the gene expression pattern of a patient to determine prognosis of colon cancer is through the use of a Cox hazard analysis program. Most preferably, the analysis is conducted using S-Plus software (commercially available from Insightful Corporation). Using such methods, a gene expression profile is compared to that of a profile that confidently represents relapse (i.e., expression levels for the combination of genes in the profile is indicative of relapse). The Cox hazard model with the established threshold is used to compare the similarity of the two profiles (known relapse versus patient) and then determines whether the patient profile exceeds the threshold. If it does, then the patient is classified as one who will relapse and is accorded treatment such as adjuvant therapy. If the patient profile does not exceed the threshold then they are classified as a non-relapsing patient. Other analytical tools can also be used to answer the same question such as, linear discriminate analysis, logistic regression and neural network approaches.

Numerous other well-known methods of pattern recognition are available. The following references provide some examples:

Weighted Voting:
Golub, T R., Slonim, D K., Tamaya, P., Huard, C., Gaasenbeek, M., Mesirov, J P., Coller, H., Loh, L., Downing, J R., Caligiuri, M A., Bloomfield, C D., Lander, E S. Molecular classification of cancer: class discovery and class prediction by gene expression monitoring. Science 286:531-537, 1999

Support Vector Machines:
Su, A I., Welsh, J B., Sapinoso, L M., Kern, S G., Dimitrov, P., Lapp, H., Schultz, P G., Powell, S M., Moskaluk, C A., Frierson, H F. Jr., Hampton, G M. Molecular classification of human carcinomas by use of gene expression signatures. Cancer Research 61:7388-93, 2001
Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001

K-Nearest Neighbors:
Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001

Correlation Coefficients:
van't Veer L J, Dai H, van de Vijver M J, He Y D, Hart A A, Mao M, Peterse H L, van der Kooy K, Marton M J, Witteveen A T, Schreiber G J, Kerkhoven R M, Roberts C, Linsley P S, Bernards R, Friend S H. Gene expression profiling predicts clinical outcome of breast cancer. Nature. 2002 January 31;415(6871):530-6.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional markers such as serum protein markers (e.g., Cancer Antigen 27.29 (CA 27.29)). A range of such markers exists including such analytes as CA 27.29. In one such method, blood is periodically taken from a treated patient and then subjected to an enzyme immunoassay for one of the serum markers described above. When the concentration of the marker suggests the return of tumors or failure of therapy, a sample source amenable to gene expression analysis is taken. Where a suspicious mass exists, a fine needle aspirate is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting breast cancer.

Kits made according to the invention include formatted assays for determining the gene expression profiles. These can include all or some of the materials needed to conduct the assays such as reagents and instructions.

The invention is further illustrated by the following non-limiting examples.

### EXAMPLES

Genes analyzed according to this invention are typically related to full-length nucleic acid sequences that code for the production of a protein or peptide. One skilled in the art will recognize that identification of full-length sequences is not necessary from an analytical point of view. That is, portions of the sequences or ESTs can be selected according to well-known principles for which probes can be designed to assess gene expression for the corresponding gene.

### Example 1

### Sample Handling and Microarray Work

Fresh frozen tissue samples were collected from patients who had surgery for breast tumors. The samples that were used were from 286 breast cancer patients staged according to standard clinical diagnostics and pathology. Clinical outcomes of the patients were known. Characteristics of the samples and the patients from whom they were obtained are shown in Table 1. None of the patients from whom the samples were obtained received adjuvant or neo-adjuvant systemic therapy. Radiotherapy was applied to 248 patients (87%). Lymph node negativity was based on pathological examination. Estrogen Receptor (ER) and Progesterone Receptor (PgR) levels for 280 tumors were measured by standard pathology tests (EIA, IHC, etc.); cutoff=10 fmol/mg protein or >10% positive tumor cells. Of the 286 patients included, 104 showed evidence of distant metastasis within 5 years. Five patients died without evidence of disease and were censored at last follow-up. Eighty-three patients died after a previous relapse.

For isolation of RNA, 20 to 40 cryostat sections of 30 μm were cut from each sample, in total corresponding to approximately 100 mg of tissue. Before, in between, and after cutting the sections for RNA isolation, 5 μm sections were cut for hematoxylin and eosin staining to confirm the presence of tumor cells. Total RNA was isolated with RNAzol B (Campro Scientific, Veenendaal, Netherlands), and dissolved in DEPC (0.1%)-treated H₂O. About 2 ng of total RNA was resuspended in 10 ul of water and 2 rounds of the T7 RNA polymerase based amplification were performed to yield about 50 ug of amplified RNA.

Total RNA samples were only used if analysis by Agilent BioAnalyzer showed clear 18S and 28S peaks with no minor peaks presents and if the area under 28S and 18S bands was greater than 15% of total RNA area. Additionally, selection criteria included a 28S/18S ratio between 1.2 and 2.0. Biotinylated targets were prepared by using published methods (Affymetrix, CA) (24) and hybridized to Affymetrix oligonucleotide microarray U133a GeneChip containing a total of 22,000 probe sets. Arrays were scanned by using the standard Affymetrix protocol. For subsequent analysis, each probe set was considered as a separate gene. Expression values for each gene were calculated by using Affymetrix GeneChip analysis software MAS 5.0. Chips were rejected if the average intensity was less than 40 or if the background signal exceeded 100. In order to normalize the chip signals, all probe sets were scaled to a target intensity of 600 and scale mask files were not selected.

TABLE 1
Clinical and Pathological Characteristics of Patients
and Their Tumors

| | All | ER-positive | ER-negative | Validation |
|---|---|---|---|---|
| Characteristics | patients | training set | training set | set |
| Number | 286 | 80 | 35 | 171 |
| Age (mean ± SD) | 54 ± 12 | 54 ± 13 | 54 ± 13 | 54 ± 12 |
| ≦40 yr | 36 (13%) | 12 (15%) | 3 (9%) | 21 (12%) |
| 41-55 yr | 129 (45%) | 30 (38%) | 17 (49%) | 82 (48%) |
| 56-70 yr | 89 (31%) | 28 (35%) | 11 (31%) | 50 (29%) |
| >70 yr | 32 (11%) | 10 (13%) | 4 (11%) | 18 (11%) |
| Menopausal status | | | | |
| Premenopausal | 139 (49%) | 39 (49%) | 16 (46%) | 84 (49%) |
| Postmenopausal | 147 (51%) | 41 (51%) | 19 (54%) | 87 (51%) |
| Tumor size | | | | |
| T1 (<2 cm) | 146 (51%) | 38 (48%) | 14 (40%) | 94 (55%) |
| T2 (2-5 cm) | 131 (46%) | 41 (51%) | 19 (54%) | 72 (42%) |
| T3/4 (>5 cm) | 8 (3%) | 1 (1%) | 2 (6%) | 5 (3%) |
| Grade | | | | |
| Poor | 148 (52%) | 37 (46%) | 24 (69%) | 87 (51%) |
| Moderate | 42 (15%) | 12 (15%) | 3 (9%) | 27 (16%) |
| Good | 7 (2%) | 2 (3%) | 2 (6%) | 3 (2%) |
| Unknown | 89 (31%) | 29 (36%) | 6 (17%) | 54 (32%) |
| ER | | | | |
| Positive | 205 (72%) | 80 (100%) | 0 (0%) | 125 (73%) |
| Negative | 75 (26%) | 0 (0%) | 35 (100%) | 40 (23%) |
| PgR | | | | |
| Positive | 165 (58%) | 59 (74%) | 5 (14%) | 101 (59%) |
| Negative | 105 (37%) | 19 (24%) | 29 (83%) | 57 (33%) |
| Metastasis <5 years | | | | |
| Yes | 104 (36%) | 30 (38%) | 18 (51%) | 56 (33%) |
| No | 182 (64%) | 50 (63%) | 17 (49%) | 115 (67%) |

ER positive and PgR positive: >10 fmol/mg protein or >10% positive tumor cells.

### Example 2

### Statistical Analysis

Gene expression data were first subjected to a filter that included only genes called “present” in 2 or more samples. Of the 22,000 genes considered, 17,819 passed this filter and were used for hierarchical clustering. Prior to the clustering, each gene was divided by its median expression level in the patients to minimize the effect of the magnitude of expression of genes, and group together genes with similar patterns of expression in the clustering analysis. Average linkage hierarchical clustering was conducted on both the genes and the samples by using GeneSpring 6.0 software to identify patient subgroups with distinct genetic profiles.

In order to identify gene markers that can best discriminate between the patients who developed a distant metastasis and the ones who remained metastasis-free within 5 years, two supervised class prediction approaches were used. In the first approach all the 286 patients were divided into a training set of 80 patients and a testing set of 206 patients. The training set was used to select gene markers and to build a prognostic signature. The testing set was used for independent validation. In the second approach, the patients were first placed into one of the two subgroups stratified by ER status. Those with an ER>10 were placed in one group (ER positive; 211 patients) and those with an ER less than or equal to 10 were placed in a separate subgroup (ER negative; 75 patients). ER cutoff establishment is discussed in more detail below.

Each patient subgroup was then analyzed separately in order to select markers. The patients in the ER-positive subgroup were divided into a training set of 80 patients and a testing set of 131 patients (125 patients with ER levels above 10 and 6 patients with unknown ER levels). The patients in the ER-negative subgroup were divided into a training set of 35 patients and a testing set of 40 patients. The training set was used to select gene markers. The markers selected from each subgroup were combined to form a single signature to predict tumor metastasis for ER-positive and ER-negative patients as a whole in a subsequent independent validation. The sample size of the training set was determined by a re-sampling method to ensure its statistical confidence level.

The following statistical methods were used to analyze the training set in order to select gene markers. First, univariate Cox proportional hazards regression was used to identify genes whose expression levels were correlated with the length of DMFS. In order to minimize the effect of multiple testing, the Cox model was performed with bootstrapping of the patients in the training set. Genes were ranked by the average p value of the Cox regression analysis. To construct a multiple gene signature, combinations of gene markers were tested by adding one gene at a time according to the rank order. Receiver Operator Characteristic (ROC) analysis was performed to calculate the area under the curve (AUC) for each signature with increasing number of genes, and the number of genes was determined when the increase of AUC starts to plateau.

The relapse score was used to determine each patient's risk of distant metastasis. The score was defined as the linear combination of weighted expression signals with the standardized Cox regression coefficient as the weight.

$$Relapse Score = A \cdot I + {\sum }_{i = 1}^{60} I \cdot {w}_{i} {x}_{i} + B \cdot ( 1 - I ) + {\sum }_{j = 1}^{16} ( 1 - I ) \cdot {w}_{j} {x}_{j} where$$

$$I = \{ \begin{matrix}1 & if ER level > 10 \\ 0 & if ER level \leq 10\end{matrix}$$

A and B are constants
wᵢ is the standardized Cox regression coefficient
xᵢ is the expression value in log 2 scale
   The gene signature and the cutoff were validated in the testing set. ROC analysis was performed for the signature. Kaplan-Meier survival plots and log-rank tests were used to assess the differences in time to distant metastasis of the predicted high and low risk groups. Sensitivity was defined as the percent of the distant metastasis patients that were predicted correctly by the gene signature, and specificity was defined as the percent of the patients free of distant recurrence that were predicted as being free of recurrence by the gene signature. Odds ratio (OR) was calculated as the ratio of the probabilities of distant metastasis between the predicted relapse patients and the predicted relapse-free patients.

Univariate and multivariate analyses using the Cox proportional hazard regression were performed on the individual clinical parameters of the patients and the combination of the clinical parameters and the gene signature. The hazard ratio (HR) and its 95% confidence interval (CI) were derived from these results. All the statistical analyses were performed using S-Plus 6 software (Insightful, VA).

The validation group of 171 patients, with 125 ER-positive and 40 ER-negative tumors combined (6 patients with unknown ER status), was not different from the total group of 286 patients with respect to any of the patients or tumor characteristics (for all factors the p value was >0.2).

Unsupervised hierarchical clustering analysis enabled a grouping of the 286 patients on the basis of the similarities of their expression profiles measured over 17,000 informative genes. Two distinct subgroups of patients were found in the clustering result. Further examination of this result showed that the classification is highly correlated to the ER status of the patients. Using the biochemical analysis on ER, 205 patients showed a ER level above 10 and were classified as ER positive tumor while 75 patients gave a ER level below 10 and were classified as ER negative tumor. Based on the result of the clustering analysis, patients were grouped as ER positive samples and as ER negative samples. A chi square test produced a p value of 2.27×10⁻²³, indicating that the classification on ER status by the two methods was highly consistent.

Using the first approach to identifying gene markers described above, thirty-five genes (SEQ ID NO 1-35) were selected from 80 patients in the training set and a Cox model to predict the occurrence of distant metastasis was built. The performance of this 35-gene signature on the testing set of 206 patients gave a sensitivity of 90% (60 of 67) and a specificity of 29% (41 of 139). This performance indicates that the patients that have the RS above the threshold of the prognostic signature have a 3.6-fold odds ratio (95% CI: 1.5-8.5; p=0.043) to develop tumor metastasis within 5 years compared with those that have the relapse score below the threshold of the prognostic signature.

In the second approach to identifying gene markers described above via division of patient subgroup based on ER status, seventy-six genes were selected from the patients in the training sets. Sixty genes were selected for the ER-positive group (SEQ ID NO 36-95). Sixteen genes were selected for the ER-negative group (SEQ ID NO 96-111), a patient group which previously had no genetic basis for prognosis. Taking together the selected genes (SEQ ID NO 36-111) and ER, a Cox model to predict patient recurrence was built for the LNN patients as a whole, i.e., for ER-positive and ER-negative patients combined. The 76-gene portfolio (and its component 16 and 60 gene portfolios) is summarized in Table 2.

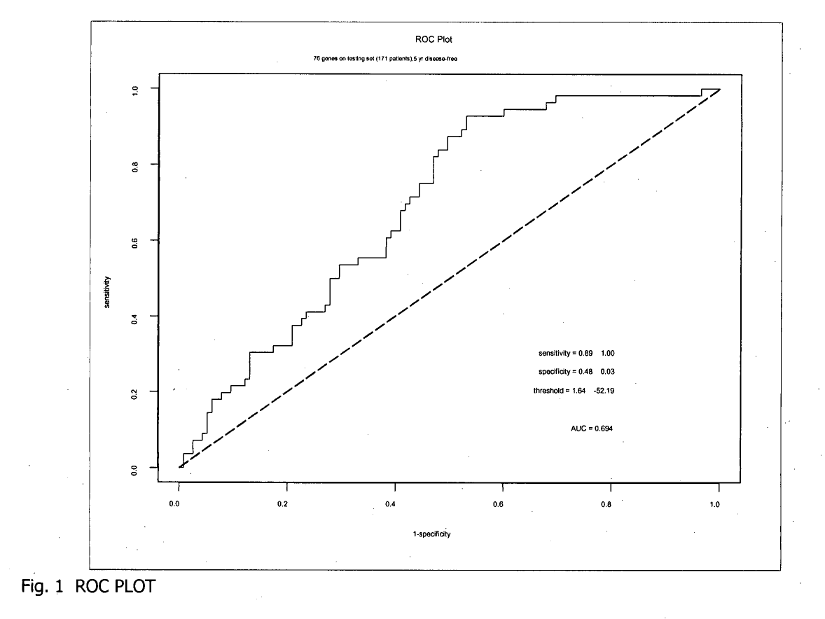

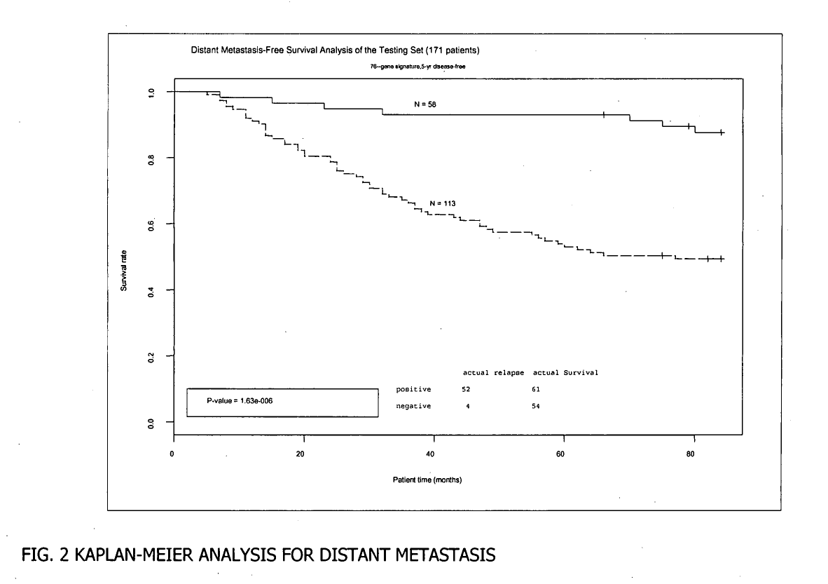

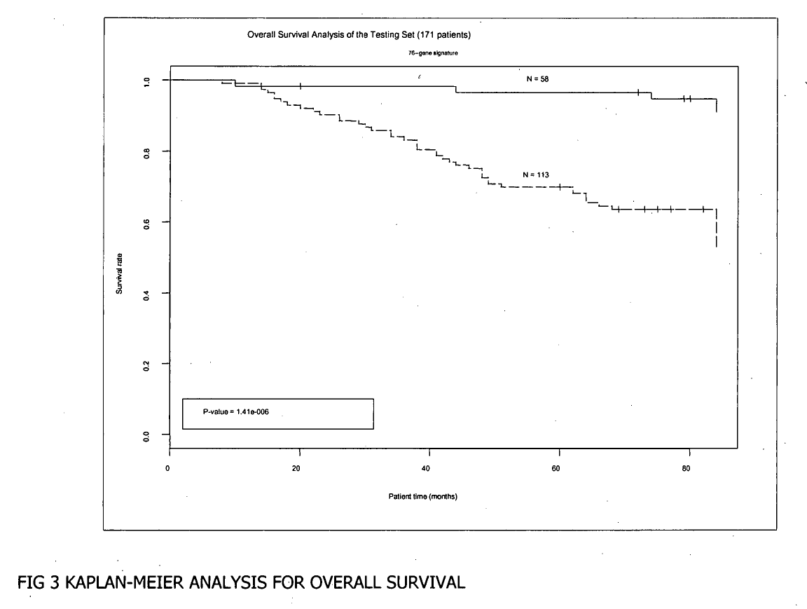

A ROC curve was produced using the 171 patients in the testing set and used AUC to assess the performance of the signature. The 76-gene predictor gave an AUC value of 0.68 (FIG. 1). The validation result of the 76-gene prognostic signature displayed a performance on the testing set with a sensitivity of 93% (52 of 56) and a specificity of 47% (54 of 115). This performance indicates that the patients that have the relapse score above the threshold of the prognostic signature have a 11.5-fold odds ratio (95% CI: 3.9-33.9; p<0.0001) to develop a distant metastasis within 5 years compared with those that have the relapse score below the threshold of the prognostic signature. In addition, the Kaplan-Meier analyses for distant metastasis free survival (DMFS) and overall survival (OS) as a function of the 76 gene-signature showed highly significant differences in the time to metastasis (FIG. 2) (HR: 5.50, 95% CI: 2.51-12.1) and death (FIG. 3) (HR: 6.93, 95% CI: 2.76-11.4) between the group predicted with good prognosis and the group predicted with poor prognosis (p value of <0.0001 for both). At 60 and 80 months, the respective differences in DMFS between the good and poor prognosis groups were 40% (93% vs. 53%) and 38% (88% vs. 50%) in the analysis of DMFS, and 27% (97% vs. 70%) and 31% (95% vs. 64%) in the analysis of OS (FIG. 3).

In additional analyses on the validation set of 171 LNN patients, the performance of the 76-gene signature was evaluated separately in the analysis of DMFS and OS for 84 premenopausal, 87 postmenopausal patients, and the 79 patients with a tumor size ranging from 10 to 20 mm representing a group of patients that are difficult to predict outcome based on clinical data. The results show that the signature predicts early metastasis and death for both premenopausal (HR: 9.0, 95% CI: 2.14-38.1, p=0.0027; and HR: 8.7, 95% CI: 2.07-37, p=0.0032, respectively) and postmenopausal patients (HR: 4.0, 95% CI: 1.57-10.4, p=0.0039; and HR: 3.84, 95% CI: 1.49-9.89, p=0.0053). Furthermore, for the patients with a tumor size between 10 and 20 mm the 76-gene signature was a strong prognostic factor in the analysis for DMFS (HR: 13.2, 95% Cl: 3.13-55.4; p=0.0004) and OS (HR: 12.6, 95% CI: 3.0-53.2, p=0.0005). Patients with this tumor size had been among the most difficult for physicians to prognosticate.

The results of the univariate and multivariate Cox regression analysis are summarized in Table 3. In the univariate result, besides the 76-gene signature only grade of differentiation was statistically significant and moderate/good differentiation was associated with favorable DMFS. In the multivariate Cox proportional hazards regression the estimated HR for the occurrence of tumor metastasis within 5 years is 6.38 (95% CI: 2.67-15.3; p=3×10⁻⁵) indicating that the 76-gene set represents an independent prognostic signature that is strongly associated with a higher risk of tumor metastasis and death. Portfolios can also be made using combinations of genes selected from within the 76-gene signature. Smaller gene expression portfolios would necessarily have lessened predictive values but can be useful if the clinician is willing to accept lower sensitivity and/or specificity. This can be particularly beneficial if the prognostic employs the smaller portfolio in combination with other diagnostic or prognostic tools or portfolios.

TABLE 2
Gene Expression Portfolio

| | Std. Cox | Cox | Gene |
|---|---|---|---|
| SEQ ID NO. | coefficient | p value | description |
| Seq ID No. 36 | −3.830 | 0.00005 | gb: AF123759.1 /DEF = Homo sapiens putative transmembrane |
| | | | protein (CLN8) mRNA, complete cds. |
| Seq ID No. 37 | −3.865 | 0.00001 | gb: NM_016548.1 /DEF = Homo sapiens golgi membrane. |
| | | | protein GP73 (LOC51280) |
| Seq ID No. 38 | 3.630 | 0.00002 | gb: NM_020470.1 /DEF = Homo sapiens putative transmembrane |
| | | | protein; homolog of yeast Golgi membrane protein Yif1p |
| Seq ID No. 39 | −3.471 | 0.00016 | gb: NM_001562.1 /DEF = Homo sapiens interleukin |
| | | | 18 (interferon-gamma-inducing factor) (IL18) |
| Seq ID No. 40 | 3.506 | 0.00008 | Consensus includes gb: BE748755 /heterochromatin- |
| | | | like protein 1 |
| Seq ID No. 41 | −3.476 | 0.00001 | gb: BC002671.1 /DEF = Homo sapiens, dual specificity |
| | | | phosphatase 4 |
| Seq ID No. 42 | 3.392 | 0.00006 | gb: NM_002710.1 /DEF = Homo sapiens protein phosphatase 1, |
| | | | catalytic subunit, gamma isoform (PPP1CC) |
| Seq ID No. 43 | −3.353 | 0.00080 | gb: NM_006720.1 /DEF = Homo sapiens actin binding LIM |
| | | | protein 1 (ABLIM), transcript variant ABLIM-s |
| Seq ID No. 44 | −3.301 | 0.00038 | gb: AF114013.1 /DEF = Homo sapiens tumor necrosis |
| | | | factor-related death ligand-1gamma |
| Seq ID No. 45 | 3.101 | 0.00033 | Consensus includes gb: AI636233 five-span transmembrane |
| | | | protein M83 |
| Seq ID No. 46 | −3.174 | 0.00128 | gb: NM_000064.1 /DEF = Homo sapiens complement |
| | | | component 3 (C3) |
| Seq ID No. 47 | 3.083 | 0.00020 | gb: NM_017760.1 /DEF = Homo sapiens hypothetical |
| | | | protein FLJ20311 |
| Seq ID No. 48 | 3.336 | 0.00005 | gb: NM_013279.1 /DEF = Homo sapiens chromosome |
| | | | 11open reading frame 9 (C11ORF9) |
| Seq ID No. 49 | −3.054 | 0.00063 | Consensus includes gb: AL523310 putative translation |
| | | | initiation factor |
| Seq ID No. 50 | −3.025 | 0.00332 | gb: AF220152.2 /DEF = Homo sapiens TACC2 Mrna |
| Seq ID No. 51 | 3.095 | 0.00044 | gb: NM_005496.1 /DEF = Homo sapiens chromosome- |
| | | | associated polypeptide C (CAP-C) |
| Seq ID No. 52 | −3.175 | 0.00031 | gb: NM_013936.1 /DEF = Homo sapiens olfactory receptor, |
| | | | family 12, subfamily D, member 2 (OR12D2) |
| Seq ID No. 53 | −3.082 | 0.00086 | gb: AF125507.1 /DEF = Homo sapiens origin recognition |
| | | | complex subunit 3 (ORC3) |
| Seq ID No. 54 | 3.058 | 0.00016 | gb: NM_014109.1 /DEF = Homo sapiens PRO2000 protein |
| | | | (PRO2000) |
| Seq ID No. 55 | 3.085 | 0.00009 | gb: AL136877.1 /SMC4 (structural maintenance of |
| | | | chromosomes 4, yeast)-like 1 /FL = gb: AB019987.1 |
| | | | gb: NM_005496.1 gb: AL136877.1 |
| Seq ID No. 56 | −2.992 | 0.00040 | gb: NM_014796.1 /DEF = Homo sapiens KIAA0748 |
| | | | gene product (KIAA0748) |
| Seq ID No. 57 | −2.791 | 0.00020 | gb: NM_001394.2 /DEF = Homo sapiens dual specificity phosphatase |
| | | | 4 (DUSP4) |
| Seq ID No. 58 | −2.948 | 0.00039 | Consensus includes gb: AI493245 /CD44 antigen |
| | | | (homing function and Indian blood group system) |
| Seq ID No. 59 | 2.931 | 0.00020 | gb: NM_005030.1 /DEF = Homo sapiens polo |
| | | | (Drosophia)-like kinase (PLK) |
| Seq ID No. 60 | −2.896 | 0.00052 | gb: NM_006314.1 /DEF = Homo sapiens connector enhancer |
| | | | of KSR-like (Drosophila kinase suppressor of ras) (CNK1) |
| Seq ID No. 61 | 2.924 | 0.00050 | gb: NM_003543.2 /DEF = Homo sapiens H4 histone family, |
| | | | member H (H4FH) |
| Seq ID No. 62 | 2.915 | 0.00055 | gb: NM_004111.3 /DEF = Homo sapiens flap structure- |
| | | | specific endonuclease 1 (FEN1) |
| Seq ID No. 63 | −2.968 | 0.00099 | gb: NM_004470.1 /DEF = Homo sapiens FK506-binding |
| | | | protein 2 (13 kD) (FKBP2) |
| Seq ID No. 64 | 2.824 | 0.00086 | gb: BC005978.1 /DEF = Homo sapiens, karyopherin alpha |
| | | | 2 (RAG cohort 1, importin alpha 1) |
| Seq ID No. 65 | −2.777 | 0.00398 | gb: NM_015997.1 /DEF = Homo sapiens CGI-41 protein |
| | | | (LOC51093) |
| Seq ID No. 66 | −2.635 | 0.00160 | gb: NM_030819.1 /DEF = Homo sapiens hypothetical |
| | | | protein MGC11335 (MGC11335) |
| Seq ID No. 67 | −2.854 | 0.00053 | gb: BC006155.1 /DEF = Homo sapiens, clone |
| | | | MGC: 13188 |
| Seq ID No. 68 | 2.842 | 0.00051 | gb: NM_024629.1 /DEF = Homo sapiens hypothetical |
| | | | protein FLJ23468 (FLJ23468) |
| Seq ID No. 69 | −2.835 | 0.00033 | Consensus includes gb: AA772093 /neuralized (Drosophila)- |
| | | | like /FL = gb: U87864.1 gb: AF029729.1 gb: NM_004210.1 |
| Seq ID No. 70 | 2.777 | 0.00164 | gb: NM_007192.1 /DEF = Homo sapiens chromatin-specific |
| | | | transcription elongation factor, 140 kDa subunit (FACTP140) |
| Seq ID No. 71 | −2.759 | 0.00222 | Consensus includes gb: U07802 /DEF = Human |
| | | | Tis11d gene |
| Seq ID No. 72 | −2.745 | 0.00086 | gb: NM_001175.1 /DEF = Homo sapiens Rho GDP |
| | | | dissociation inhibitor (GDI) beta (ARHGDIB) |
| Seq ID No. 73 | 2.790 | 0.00049 | gb: NM_002803.1 /DEF = Homo sapiens proteasome |
| | | | (prosome, macropain) 26S subunit, ATPase, 2 (PSMC2) |
| Seq ID No. 74 | 2.883 | 0.00031 | gb: NM_017612.1 /DEF = Homo sapiens hypothetical |
| | | | protein DKFZp434E2220 (DKFZp434E2220) |
| Seq ID No. 75 | −2.794 | 0.00139 | Consensus includes gb: R39094 /KIAA1085 protein |
| Seq ID No. 76 | −2.743 | 0.00088 | gb: BC004372.1 /DEF = Homo sapiens, Similar to CD44 |
| | | | antigen (homing function and Indian blood group system) |
| Seq ID No. 77 | −2.761 | 0.00164 | Consensus includes gb: AL117652.1 /DEF = Homo |
| | | | sapiens mRNA |
| Seq ID No. 78 | −2.831 | 0.00535 | gb: NM_006416.1 /DEF = Homo sapiens solute carrier |
| | | | family 35 (CMP-sialic acid transporter), member 1 (SLC35A1) |
| Seq ID No. 79 | 2.659 | 0.00073 | gb: NM_004702.1 /DEF = Homo sapiens cyclin |
| | | | E2 (CCNE2) |
| Seq ID No. 80 | −2.715 | 0.00376 | Consensus includes gb: BF055474 /putative zinc |
| | | | finger protein NY-REN-34 antigen |
| Seq ID No. 81 | 2.836 | 0.00029 | gb: NM_006596.1 /DEF = Homo sapiens polymerase |
| | | | (DNA directed), theta (POLQ) |
| Seq ID No. 82 | −2.687 | 0.00438 | Consensus includes gb: AF041410.1 /DEF = Homo sapiens |
| | | | malignancy-associated protein |
| Seq ID No. 83 | −2.631 | 0.00226 | gb: M23254.1 /DEF = Human Ca2-activated neutral |
| | | | protease large subunit (CANP) |
| Seq ID No. 84 | −2.716 | 0.00089 | Consensus includes gb: AV693985 /ets variant |
| | | | gene 2 |
| Seq ID No. 85 | 2.703 | 0.00232 | gb: NM_017859.1 /DEF = Homo sapiens hypothetical |
| | | | protein FLJ20517 (FLJ20517) |
| Seq ID No. 86 | −2.641 | 0.00537 | Consensus includes gb: AV713720 /Homo sapiens |
| | | | mRNA for LST-1N protein |
| Seq ID No. 87 | −2.686 | 0.00479 | Consensus includes gb: AI057637 /Hs.234898 ESTs, |
| | | | Weakly similar to 2109260A B cell growth factor H. sapiens |
| Seq ID No. 88 | −2.654 | 0.00363 | Consensus includes gb: U90030.1 /DEF = Homo sapiens |
| | | | bicaudal-D (BICD) mRNA, alternatively spliced, partial cds. |
| Seq ID No. 89 | 2.695 | 0.00095 | gb: NM_001958.1 /DEF = Homo sapiens eukaryotic translation |
| | | | elongation factor 1 alpha 2 (EEF1A2) |
| Seq ID No. 90 | −2.758 | 0.00222 | Consensus includes gb: BF055311 /hypothetical protein |
| Seq ID No. 91 | 2.702 | 0.00084 | Consensus includes gb: AL133102.1 /DEF = Homo |
| | | | sapiens mRNA; cDNA DKFZp434C1722 |
| Seq ID No. 92 | −2.694 | 0.00518 | gb: AF114012.1 /DEF = Homo sapiens tumor necrosis |
| | | | factor-related death ligand-1beta mRNA |
| Seq ID No. 93 | 2.711 | 0.00049 | Consensus includes gb: AK001280.1 /DEF = Homo |
| | | | sapiens cDNA FLJ10418 fis, clone NT2RP1000130, |
| | | | moderately similar to HEPATOMA-DERIVED GROWTH FACTOR. |
| Seq ID No. 94 | −2.771 | 0.00156 | gb: NM_004659.1 /DEF = Homo sapiens matrix |
| | | | metalloproteinase 23A (MMP23A) |
| Seq ID No. 95 | 2.604 | 0.00285 | gb: BC006325.1 /DEF = Homo sapiens, G-2 and |
| | | | S-phase expressed 1 |
| Seq ID No. 96 | −3.495 | 0.00011 | gb: NM_022841.1 /DEF = Homo sapiens hypothetical |
| | | | protein FLJ12994 (FLJ12994) |
| Seq ID No. 97 | 3.224 | 0.00036 | Consensus includes gb: X16468.1 /DEF = Human |
| | | | mRNA for alpha-1 type II collagen. |
| Seq ID No. 98 | −3.225 | 0.00041 | gb: NM_005256.1 /DEF = Homo sapiens growth |
| | | | arrest-specific 2 (GAS2) |
| Seq ID No. 99 | −3.145 | 0.00057 | Consensus includes gb: AK021842.1 /DEF = Homo |
| | | | sapiens cDNA FLJ11780 fis, clone HEMBA1005931, |
| | | | weakly similar to ZINC FINGER PROTEIN 83. |
| Seq ID No. 100 | −3.055 | 0.00075 | Consensus includes gb: D89324 /DEF = Homo sapiens |
| | | | DNA for alpha (1,31,4) fucosyltransferase |
| Seq ID No. 101 | −3.037 | 0.00091 | gb: NM_017534.1 /DEF = Homo sapiens myosin, heavy |
| | | | polypeptide 2, skeletal muscle, adult (MYH2) |
| Seq ID No. 102 | −3.066 | 0.00072 | gb: U57059.1 /DEF = Homo sapiens Apo-2 ligand mRNA |
| Seq ID No. 103 | 3.060 | 0.00077 | gb: BC000596.1 /DEF = Homo sapiens, Similar to |
| | | | ribosomal protein L23a, clone MGC: 2597 |
| Seq ID No. 104 | −2.985 | 0.00081 | gb: NM_018558.1 /DEF = Homo sapiens gamma- |
| | | | aminobutyric acid (GABA) receptor, theta (GABRQ) |
| Seq ID No. 105 | −2.983 | 0.00104 | gb: NM_006437.2 /DEF = Homo sapiens ADP- |
| | | | ribosyltransferase (NAD+; poly (ADP-ribose) polymerase)- |
| | | | like 1 (ADPRTL1) |
| Seq ID No. 106 | −3.022 | 0.00095 | gb: NM_014042.1 /DEF = Homo sapiens DKFZP564M082 |
| | | | protein (DKFZP564M082) |
| Seq ID No. 107 | −3.054 | 0.00082 | gb: NM_030766.1 /DEF = Homo sapiens apoptosis |
| | | | regulator BCL-G (BCLG) |
| Seq ID No. 108 | −3.006 | 0.00098 | gb: BC001233.1 /DEF = Homo sapiens, Similar to |
| | | | KIAA0092 gene product, clone MGC: 4896 |
| Seq ID No. 109 | −2.917 | 0.00134 | Consensus includes gb: AL137162 /Contains a novel |
| | | | gene and the 5 part of a gene for a novel protein similar |
| | | | to X-linked ribosomal protein 4 (RPS4X) |
| Seq ID No. 110 | −2.924 | 0.00149 | gb: M55580.1 /DEF = Human spermidinespermine |
| | | | N1-acetyltransferase |
| Seq ID No. 111 | −2.882 | 0.00170 | Consensus includes gb: AB014607.1 /DEF = Homo |
| | | | sapiens mRNA for KIAA0707 protein |

1. A method of assessing breast cancer status comprising identifying differential modulation in a combination of genes selected from the group consisting of SEQ ID NO 1-111.

2. The method of claim 1 wherein the expression pattern of the genes is compared to an expression pattern indicative of a relapse patient.

3. The method of claim 2 wherein the comparison of expression patterns is conducted with pattern recognition methods.

4. The method of claim 3 wherein the pattern recognition methods include the use of a Cox proportional hazards analysis.

5. The method of claim 1 conducted on primary tumor sample.

6. The method of claim 1 wherein the combination includes all of the genes corresponding to SEQ ID NO 1-35.

7. The method of claim 1 wherein the combination includes all of the genes corresponding to SEQ ID NO 36-95.

8. The method of claim 7 used to provide a prognosis for ER negative patients.

9. The method of claim 1 wherein the combination includes all of the genes corresponding to SEQ ID NO 96-111.

10. The method of claim 9 used to provide a prognosis for ER positive patients.

11. The method of claim 1 wherein the combination includes all of the genes corresponding to SEQ ID NO 36-111.

12. The method of claim 1 wherein there is at least a 2 fold difference in the expression of the modulated genes.

13. The method of claim 1 wherein the p-value indicating differential modulation is less than 0.05.

14. The method of claim 1 further comprising a breast diagnostic that is not genetically based.

15. The method of claim 14 wherein said diagnostic is ER status.

16. A prognostic portfolio comprising isolated nucleic acid sequences, their complements, or portions thereof of a combination of genes selected from the group consisting of SEQ ID NO 1-111.

17. The portfolio of claim 16 wherein the combination includes all of the genes corresponding to SEQ ID NO 36-95.

18. The portfolio of claim 17 used to provide a prognosis for ER positive patients.

19. The portfolio of claim 16 wherein the combination includes all of the genes corresponding to SEQ ID NO 96-111.

20. The portfolio of claim 19 used to provide a prognosis for ER negative patients.

21. The portfolio of claim 16 wherein the combination includes all of the genes corresponding to SEQ ID NO 36-111.

22. The portfolio of claim 16 in a matrix suitable for identifying the differential expression of the genes contained therein.

23. The portfolio of claim 22 wherein said matrix is employed in a microarray.

24. The portfolio of claim 23 wherein said microarray is a cDNA microarray.

25. The portfolio of claim 23 wherein said microarray is an oligonucleotide microarray.

26. A kit for determining the prognosis of a breast cancer patient comprising materials for detecting isolated nucleic acid sequences, their compliments, or portions thereof of a combination of genes selected from the group consisting of SEQ ID NO 1-111.

27. The kit of claim 26 wherein all of the genes correspond to SEQ ID NO 36-95.

28. The kit of claim 26 wherein all of the genes correspond to SEQ ID NO 96-111.

29. The kit of claim 26 wherein all of the genes correspond to SEQ ID NO 36-111.

30. The kit of claim 26 further comprising reagents for conducting a microarray analysis.

31. The kit of claim 26 further comprising a medium through which said nucleic acid sequences, their compliments, or portions thereof are assayed.

32. Articles for assessing breast cancer status comprising materials for identifying nucleic acid sequences, their complements, or portions thereof of a combination of genes selected from the group consisting of SEQ ID NO 1-111.

33. The articles of claim 32 wherein all of the genes correspond to SEQ ID NO 36-95.

34. The articles of claim 32 wherein all of the genes correspond to SEQ ID NO 96-111.

35. The articles of claim 32 wherein all of the genes correspond to SEQ ID NO 35-111.

36. A method of treating a breast cancer patient comprising characterizing the patient as high risk for recurrence or not based on the expression of a combination of genes selected from the group consisting of SEQ ID NO 1-111 and treating the patient with adjuvant therapy if they are a high risk patient.

37. The method of claim 36 wherein all of the genes correspond to SEQ ID NO 36-95.

38. The method of claim 36 wherein all of the genes correspond to SEQ ID NO 96-111.

39. The method of claim 36 wherein all of the genes correspond to SEQ ID NO 36-111.
